(19) United States
(12) Patent Application Publication
(10) Pub. No.: US 2022/0331005 A1
(43) Pub. Date: Oct. 20, 2022

(54) Ablation Needle with Positioning Sensor Installed on Head Portion Thereof

(71) Applicant: Zhejiang Demetics Medical Technology Co., Ltd., Hangzhou (CN)

(72) Inventor: Chaowei Chen, Hangzhou (CN)

(21) Appl. No.: 17/232,103

(22) Filed: Apr. 15, 2021

(51) Int. Cl. A61B 18/14; A61B 34/20

(52) U.S. Cl. CPC A61B 18/1477; A61B 34/20; A61B 2018/00577

(57) ABSTRACT

An ablation needle with a positioning sensor installed on a head portion thereof includes a positioning sensor, a needle body, and a handle. The positioning sensor is installed at a front end of an inner cavity of the needle body, and wires connected to the positioning sensor are led out from a rear end of the inner cavity of the needle body. The ablation needle can obtain real-time three-dimensional coordinate information and posture information of the head portion during passing thereof, without being affected by bending of the needle body; and with assistance of three-dimensional image processing software and hardware, a route of passing the ablation needle and a position of the ablation needle can be dynamically displayed in real time.

### FIELD OF THE INVENTION

This application belongs to medical device technologies, and in particular relates to an ablation needle with a positioning sensor installed on a head portion thereof.

### BACKGROUND OF THE INVENTION

Currently, there are two ways to treat tumors by ablation, which are microwave ablation and radiofrequency ablation. The key step of ablation surgery is to safely and accurately pass the head of the ablation needle into the tumor site to be treated. Blood vessels, ribs, and important organs must be avoided during the passing of the ablation needle. The existing ablation treatment devices, however, have the following defects.

(1) The passing of the ablation needle needs to be completed by a professional doctor with rich clinical experience. During the passing of the ablation needle, it is necessary to perform three-dimensional space imaging based on imaging information such as ultrasound, CT, and MRI to determine the current specific position of the ablation needle, and meanwhile blood vessels, ribs, and organs must be avoided. These are very difficult for doctors new to the ablation surgery.

(2) In practical clinical treatment, due to the complex structure of tumor tissues, the head of the ablation needle is not clearly visible in some tissues during image-guided passing of the ablation needle, which makes it difficult for doctors to judge the current position of the ablation needle.

(3) Chinese patent application CN204708979U discloses an ablation needle with a positioning device. The ablation needle uses a magnetic positioning sensor installed on a tail portion thereof to realize positioning in an electromagnetic tracking and positioning system, and then obtained parameters are corrected to obtain a three-dimensional coordinate position of a head of the ablation needle. However, in this technical solution, the magnetic positioning sensor on the ablation needle is installed on the tail portion of the ablation needle. When an ablation needle enters human tissues, it will be deformed due to the force exerted thereon, and such deformation will be different depending on different patients, different surgical positions, or different surgical techniques of different doctors. Therefore, the magnetic positioning sensor installed on the tail portion cannot accurately measure the spatial position information of the tip of the needle.

### SUMMARY OF THE INVENTION

The technical problem to be solved by the present disclosure is to overcome the shortcomings of the existing technologies and provide an ablation needle with a positioning sensor installed on a head portion thereof.

In order to solve the above technical problem, the present disclosure adopts the following technical solutions.

Provided is an ablation needle with a positioning sensor installed on a head portion thereof. The ablation needle includes a positioning sensor, a needle body, and a handle. The positioning sensor is installed at a front end of an inner cavity of the needle body, and wires connected to the positioning sensor are led out from a rear end of the inner cavity of the needle body.

As an improvement, the positioning sensor is fixed at the front end of the inner cavity of the needle body by means of a (heat-resistant) UV adhesive.

As an improvement, the positioning sensor is a magnetic positioning sensor having a maximum diameter of 0.55 mm and a maximum length of 8 mm.

As an improvement, provided are two wires. Surfaces of the wires each are covered with a (heat-resistant) insulating coating. An opening is provided on a tail portion or a side of the handle to lead out the wires, and the opening is filled with a UV adhesive to achieve fixation. The wires located outside are wrapped with a flexible insulating rubber.

As an improvement, the wires each have a maximum diameter of 0.3 mm.

Compared with the existing technologies, the present disclosure has the following advantages.

(1) The ablation needle of the present disclosure can obtain real-time three-dimensional coordinate information and posture information of the head portion during passing of the ablation needle, without being affected by bending of the needle body. With assistance of three-dimensional image processing software and hardware, a route of passing the ablation needle and a position of the ablation needle can be dynamically displayed in real time. This can provide accurate positioning information for passing of an ablation needle in a clinical ablation surgery.

(2) The present disclosure can help to avoid the defect of unclear visualization of an ablation needle in some tissues in clinical practice and thus incapability of obtaining the current accurate position of the ablation needle. This makes the surgery easier, reduces clinical risks, and decreases surgical trauma.

### BRIEF DESCRIPTION OF THE DRAWINGS

FIG. 1 is a schematic structural diagram of an ablation needle of the present disclosure.

FIG. 2 is a schematic block diagram of a positioning device.

FIG. 3 is a schematic diagram showing realization of electromagnetic tracking and positioning.

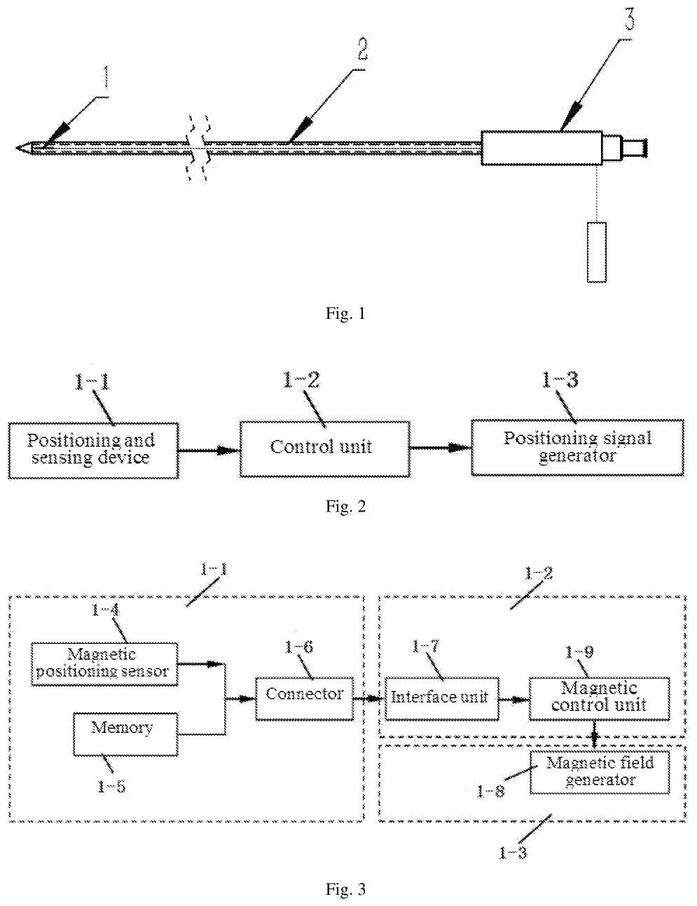

In FIG. 1, reference numeral 1 represents a positioning sensor; reference numeral 2 represents a needle body of the ablation needle; and reference numeral 3 represents a handle.

### DETAILED DESCRIPTION OF THE EMBODIMENTS

As shown in FIG. 1, an ablation needle with a positioning sensor installed on a head portion thereof provided in the present disclosure includes a positioning sensor 1, a needle body 2, and a handle 3. The positioning sensor 1 is installed at a front end of an inner cavity of the needle body 2, and may be fixed by means of a heat-resistant UV adhesive. Wires connected to the positioning sensor 1 are led out from a rear end of the inner cavity of the needle body 2. Since the rear end of the needle body 2 is installed on the handle 3, an opening may be provided on a tail portion or a side of the handle 3 to lead out the wires, and the opening is filled with a heat-resistant UV adhesive to achieve fixation. The wires located outside are wrapped with a flexible insulating rubber. Provided are two wires. Surfaces of the wires each are covered with a heat-resistant insulating coating, and the wires each have a maximum diameter of 0.3 mm.

A needle body 2 used for a commonly used microwave ablation needle is usually a titanium alloy needle tube having an outer diameter of 1.6 mm and an inner diameter of 1.2 mm, and a microwave coaxial cable used has a diameter of 0.5 mm. The positioning sensor 1 may be a magnetic positioning sensor having a maximum diameter of 0.55 mm and a maximum length of 8 mm; the wires used each have a maximum diameter of 0.3 mm, and surfaces thereof each are covered with a heat-resistant insulating coating, so that the wires are insulated from the microwave coaxial cable located in the inner cavity of the needle body 2; and the two wires are led out through the opening on the side of the handle along an inner wall, and are fixed with the handle by means of the UV adhesive. Therefore, the positioning sensor 1 in the present disclosure can be securely installed in a conventional ablation needle without changing original structural features of the needle body 2, and thus does not negatively affect functional performance of the needle body 2.

A positioning device working together with the positioning sensor 1 and technical solutions for implementing the positioning device can be found in the prior art (e.g., the Chinese patent application CN204708979 U).

The following describes specific use of the present disclosure.

Referring to FIGS. 2 and 3, a magnetic positioning device (e.g., NDI Aurora electromagnetic tracking and positioning system) includes a positioning and sensing device 1-1, a control unit 1-2, and a positioning signal generator 1-3. The control unit 1-2 controls the positioning signal generator 1-3 to generate positioning signals required for positioning. Positioning information of the positioning and sensing device 1-1 is received and analyzed by the control unit 1-2 to obtain space coordinate information of the positioning and sensing device 1-1. The positioning and sensing device 1-1 consists of a magnetic positioning sensor 1-4, a memory 1-5, and a connector 1-6. The control unit 1-2 consists of an interface unit 1-7 and a magnetic control unit 1-9. The positioning signal generator 1-3 is a magnetic field generator 1-8. The magnetic control unit 1-9 controls the magnetic field generator 1-8 to generate a magnetic field required for positioning. Positioning information of the magnetic positioning sensor 1-4 is amplified by the interface unit 1-7 through the connector 1-6 and its electrical signal is digitized. The magnetic control unit 1-9 receives and analyzes and processes information from the interface unit 1-7 to obtain coordinate information of the magnetic positioning sensor 1-4. The magnetic positioning sensor 1-4 may be a five-degree-of-freedom sensor or a six-degree-of-freedom sensor, and is directly installed on the head portion of the needle body 2 of the ablation needle (as shown in FIG. 1). In this way combined with previously obtained CT scan images, the position of the head portion of the needle in the human tissue can be learned in real time during passing of the ablation needle in the ablation.

In the ablation surgery, the magnetic positioning sensor 1-4 transmits positioning information about the head portion of the ablation needle to a connected device. With this information combined with previously scanned images, a relative three-dimensional coordinate system of the images and the ablation needle can be formed, by way of which real-time three-dimensional coordinate position information and posture information of the ablation needle can be obtained during the passing of the ablation needle.

1. An ablation needle with a positioning sensor installed on a head portion thereof, comprising a positioning sensor, a needle body, and a handle, wherein the positioning sensor is a magnetic positioning sensor installed at a front end of an inner cavity of the needle body, and wires connected to the positioning sensor are led out from a rear end of the inner cavity of the needle body.

2. The ablation needle according to claim 1, wherein the positioning sensor is fixed at the front end of the inner cavity of the needle body by means of a UV adhesive.

3. The ablation needle according to claim 1, wherein the positioning sensor has a maximum diameter of 0.55 mm and a maximum length of 8 mm.

4. The ablation needle according to claim 1, wherein provided are two wires, wherein surfaces of the wires each are covered with an insulating coating; an opening is provided on a tail portion or a side of the handle to lead out the wires, and the opening is filled with a UV adhesive to achieve fixation; and the wires located outside are wrapped with a flexible insulating rubber.

5. The ablation needle according to claim 1, wherein the wires each have a maximum diameter of 0.3 mm.
